(19) United States
(12) Patent Application Publication
(10) Pub. No.: US 2008/0051438 A1
(43) Pub. Date: Feb. 28, 2008

(54) Preventive/Therapeutic Compositions Useful for Treating Cardiovascular Diseases

(76) Inventors: Shinobu Nagahama, Tokyo (JP); Takeo Norimatsu, Tokyo (JP); Toshio Maki, Tokyo (JP); Masaharu Yasuda, Tokyo (JP); Shinsuke Tanaka, Tokyo (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/837,727

(22) Filed: Aug. 13, 2007

Related U.S. Application Data
(60) Provisional application No. 60/891,052, filed on Feb. 22, 2007.

(30) Foreign Application Priority Data
Aug. 11, 2006 (JP) 2006-220712

(51) Int. Cl. A61K 31/44; A61P 9/12

(52) U.S. Cl. 514/356

(57) ABSTRACT

The present invention provides compositions that exert a superior hypotensive action while inhibiting an increase in heart rate. The present invention provides antihypertensives and preventive/therapeutic compositions useful in the treatment of cardiovascular diseases, which contain cilnidipine and at least one angiotensin II receptor blocker(s). The present invention also provides methods for treating cardiovascular diseases by administration of said compositions.

This application claims priority under 35 U.S.C. §119 to JP2006-220712, filed Aug. 11, 2006, and U.S. Provisional Patent Application No. 60/891,052, filed on Feb. 22, 2007, both of which are incorporated by reference.

### BACKGROUND OF THE INVENTION

1. Field of the Invention

The present invention relates to preventive/therapeutic compositions useful for treating cardiovascular diseases. More specifically, it relates to antihypertensive compositions which contain cilnidipine in combination with an angiotensin II receptor blocker(s), and methods for use thereof.

2. Brief Description of the Related Art

Angiotensin II receptor blockers (ARB) specifically antagonize, or block the action of, angiotensin II receptors. This results in an inhibition of the physiological action of angiotensin II. Angiotensin II is generated as a part of the renin-angiotensin system, and has a strong hypertensive action.

Presently, calcium channel blockers (CCB) are the most widely used antihypertensives in Japan. They are recommended as the first-line agent since they have less serious side-effects and are less expensive as compared to diuretic agents.

In the guidelines for the management of hypertension in Japan (JSH2000 and JSH2004), when treatment with antihypertensives alone cannot achieve a target blood pressure, the combined administration of CCB and ARB is recommended. The combined use of CCB and ARB is a standard treatment for hypertension.

To prevent death as a result of cardiovascular disease, controlling the blood pressure of a hypertensive patient is very important, and antihypertensives are often used for this purpose. On the other hand, an increase in heart rate is known to correlate to an increase in the death rate from cardiovascular diseases (including coronary artery diseases) (The Framingham Heart Study), and controlling the heart rate is known to be highly effective. Particularly, the risk severely increases when the heart rate of the patient is 75 beats/min. or higher, and thus, it is thought to be important to decrease an especially high heart rate. (Gillman M W, et al. “American heart journal” vol. 125, No. 4, p. 1148-1154 (1993)).

Cilnidipine is a dihydropyridine CCB as well as an antihypertensive. Cilnidipine has L- and N-calcium channel blocking actions. Though many of the dihydropyridine CCBs may cause an increase in heart rate while being effective for lowering blood pressure, it has been confirmed that cilnidipine does not increase the heart rate and has a stable hypotensive effect. (Takahiro Shiokoshi, “Medical Consultation & New Remedies” vol. 41, No. 6, p. 475-481)

However, there have been no reports of the combined use of cilnidipine and ARBs, and the effects of this combined use on blood pressure and heart rate have not been clarified.

### SUMMARY OF THE INVENTION

The present invention provides compositions that exert superior hypotensive action while inhibiting the concommittant increase in heart rate; and, especially, provides compositions that exert superior hypotensive action while inhibiting an increase in the heart rate of subjects who meet the specific conditions mentioned below.

The inventors thoroughly searched in order to solve the above problem and found that a superior hypotensive action while inhibiting an increase in heart rate caused by the decrease of blood pressure can be obtained with the combined use of ARB and cilnidipine; and the further superior hypotensive action while inhibiting the increase in heart rate caused by the decrease of blood pressure can be obtained in subjects who meet specific conditions; and thus, compositions have been obtained that can effectively prevent/treat cardiovascular diseases. The present invention has been completed based on these findings.

Namely, the present invention is as follows:

It is an aspect of the present invention to provide a composition useful for the treatment or prevention of cardiovascular diseases comprising cilnidipine and at least one angiotensin II receptor blocker.

It is a further aspect of the present invention to provide the composition as described above, wherein said composition is formed by a process comprising separately formulating the cilnidipine and the at least one angiotensin II receptor blocker, and thereafter combining the cilnidipine and the at least one angiotensin II receptor blocker.

It is a further aspect of the present invention to provide the composition as described above, wherein the at least one angiotensin II receptor blocker is candesartan.

It is a further aspect of the present invention to provide the composition as described above, wherein the ratio of candesartan to cilnidipine is 0.05:1 to 5:1 by weight.

It is a further aspect of the present invention to provide the composition as described above, wherein said composition has an antihypertensive effect when administered to a subject.

It is a further aspect of the present invention to provide a method of treating or preventing cardiovascular disease comprising administering an effective amount of the composition as described above to a subject in need thereof.

It is a further aspect of the present invention to provide the method as described above, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

It is a further aspect of the present invention to provide the method as described above, wherein the at least one calcium channel blocker is nifedipine.

It is a further aspect of the present invention to provide the method as described above, wherein said subject is a human being having a heart rate of 75 beats/min. or higher.

It is a further aspect of the present invention to provide the method as described above, wherein said subject is male.

It is a further aspect of the present invention to provide a method of treating or preventing a cardiovascular disease comprising administering an effective amount of a composition comprising cilnidipine and an effective amount of a composition comprising at least one angiotensin II receptor blocker to a subject in need thereof.

It is a further aspect of the present invention to provide the method as described above, wherein said administration of a composition comprising cilnidipine and said administration of at least one angiotensin II receptor blocker is consecutive.

It is a further aspect of the present invention to provide the method as described above, wherein said subject is a human having a heart rate of 75 beats/min. or higher.

It is a further aspect of the present invention to provide the method as described above, wherein said subject is male.

It is a further aspect of the present invention to provide the method as described above, wherein said at least one angiotensin II receptor blocker is candesartan.

It is a further aspect of the present invention to provide the method as described above, wherein the ratio of candesartan to cilnidipine is 0.05:1 to 5:1 by weight.

It is a further aspect of the present invention to provide the method as described above, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

It is a further aspect of the present invention to provide the method as described above, wherein the calcium channel blocker is nifedipine.

It is a further aspect of the present invention to provide a composition useful for treatment of hypertension comprising cilnidipine and at least one angiotensin II receptor blocker.

It is a further aspect of the present invention to provide the composition as described above, wherein said composition is formed by a process comprising separately formulating the cilnidipine and the at least one angiotensin II receptor blocker, and thereafter combining the cilnidipine and the angiotensin II receptor blocker.

It is a further aspect of the present invention to provide the composition as described above, wherein the angiotensin II receptor blocker is candesartan.

It is a further aspect of the present invention to provide the composition as described above, wherein the ratio of candesartan to cilnidipine is 0.05:1 to 5:1 by weight.

It is a further aspect of the present invention to provide the composition as described above, wherein said composition has an effect on cardiovascular diseases when administered to a subject.

It is a further aspect of the present invention to provide a method of treating hypertension comprising administering an effective amount of the composition as described above to a subject in need thereof.

It is a further aspect of the present invention to provide the method as described above, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

It is a further aspect of the present invention to provide the method as described above, wherein the calcium channel blocker is nifedipine.

It is a further aspect of the present invention to provide the method as described above, wherein the subject is a human being with a heart rate of 75 beats/min. or higher.

It is a further aspect of the present invention to provide the method as described above, wherein the subject is male.

It is a further aspect of the present invention to provide a method of treating hypertension comprising administering an effective amount of a composition comprising cilnidipine and an effective amount of a composition comprising at least one angiotensin II receptor blocker to a subject in need thereof.

It is a further aspect of the present invention to provide the method as described above, wherein said administration of a composition comprising cilnidipine and said administration of at least one angiotensin II receptor blocker is consecutive.

It is a further aspect of the present invention to provide the method as described above, wherein said subject is a human having a heart rate of 75 beats/min. or higher.

It is a further aspect of the present invention to provide the method as described above, wherein said subject is male.

It is a further aspect of the present invention to provide the method as described above, wherein said at least one angiotensin II receptor blocker is candesartan.

It is a further aspect of the present invention to provide the method as described above, wherein the ratio of candesartan to cilnidipine is 0.05:1 to 5:1 by weight.

It is a further aspect of the present invention to provide the method as described above, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

It is a further aspect of the present invention to provide the method as described above, wherein the calcium channel blocker is nifedipine.

It is a further aspect of the present invention to provide a kit comprising a composition comprising cilnidipine and a composition comprising at least one angiotensin II receptor blocker.

It is a further aspect of the present invention to provide the kit as described above, wherein said at least one angiotensin II receptor blocker is candesartan.

It is a further aspect of the present invention to provide the kit as described above, wherein the ratio of candesartan to cilnidipine is 0.05:1 to 5:1 by weight.

It is a further aspect of the present invention to provide a pharmaceutical composition comprising cilnidipine and candesartan as active ingredients.

It is a further aspect of the present invention to provide the pharmaceutical composition as described above, wherein the ratio of candesartan to cilnidipine is 0.05:1 to 5:1 by weight.

The agents of the present invention have a superior hypotensive action while inhibiting an increase in heart rate and, therefore, are useful in preventing/treating cardiovascular diseases. Furthermore, the agents have a particularly excellent hypotensive action as antihypertensives in subjects who meet specific conditions.

### BRIEF DESCRIPTION OF THE DRAWINGS

FIG. 1 is a diagram of the collected cases.

FIG. 2 is a graph showing the changes in blood pressure and heart rate in the subject cases of the availability analysis.

FIG. 3 is a graph showing the changes in blood pressure and heart rate in the cases in which only ARB was administered, and then cilnidipine was singularly and additionally administered (not including other antihypertensives), i.e. consecutive administration.

FIG. 4 is a graph showing the changes in blood pressure and heart rate for each ARB agent in the cases in which only ARB was administered, and then cilnidipine was singularly and additionally administered (not including other antihypertensives), i.e. consecutive administration.

FIG. 5 is a graph showing the changes in blood pressure and heart rate for calcium channel blockers administered with ARB before switching to the combined administration of ARB and cilnidipine.

FIG. 6 is a graph showing the changes in heart rates in cases before starting the combined use in the availability analysis.

### DESCRIPTION OF THE PREFERRED EMBODIMENTS

The present invention provides antihypertensive compositions of cilnidipine and at least one angiotensin II receptor blocker, compositions of cilnidipine, and compositions of at least one angiotensin II receptor blocker.

Angiotensin II receptor blockers (hereinafter, also “ARB” or “ARBs”) exert a hypotensive action by antagonizing, or blocking the effect of angiotensin II. Angiotensin II is a known hypertensive. The antiotensin II receptor blocker prevents angiotensin II from binding to the angiotensin II receptor. The angiotensin II receptor blockers are not particularly limited, but their combined use with cilnidipine exerts a better hypotensive action than use alone, and they are less likely to induce an increase in heart rate due to this hypotensive action. Examples of ARBs include valsartan, candesartan, losartan, telmisartan, olmesartan, irbesartan, and eprosartan. Valsartan, candesartan, losartan, and telmisartan are preferable, and candesartan is particularly preferable.

Cilnidipine (2-methoxyethyl-3-phenyl-2(E)-propenyl(±)-1,4-dihydro-2,6-dimethyl-4-(3-nitrophenyl)-3,5-pyridinedicarboxylate) is a known L/N calcium channel blocker that inhibits both the L-calcium channel and N-calcium channel. Cilnidipine can be produced by known methods or may be obtained commercially. Furthermore, cilnidipine can be obtained from pharmaceutically acceptable salts, hydrates, or solvates by extraction thereof, or the like. Cilnidipine compositions may be in the form of a pharmaceutically acceptable salts, hydrates or solvates, if necessary. Pharmaceutically acceptable salts include salts of inorganic acids, e. g. hydrochloride, hydrobromide, phosphate and sulfate; and salts of organic acids, e.g. acetate, succinate, maleate, fumarate, malate and tartrate. Furthermore, a suitable optical isomer of cilnidipine may be used, if necessary.

The compositions of the present invention, whether a administered as a composition of an ARB and cilnidipine, or a cilnidipine composition and an ARB composition administered separately, have a superior hypotensive action and inhibit the increase in heart rate which can be caused by a decrease in blood pressure. Therefore, these compositions are highly useful as preventive/therapeutic agents for the treatment of cardiovascular diseases.

Cardiovascular diseases include cardiovascular disorders such as ischemic heart disease and hypertension. Examples thereof include, but are not limited to, myocardial infarction and angina pectoris (including unstable angina pectoris).

The hypotensive action of the compositions of the present invention can be enhanced. For example, the combined use of cilnidipine and at least one angiotensin II receptor blocker(s) can improve the hypotensive action of cilnidipine or the angiotensin II receptor blocker when each used alone. Additionally, the combined use can inhibit the increase in heart rate, which often happens as a side-effect to the decrease of blood pressure due to the administration of antihypertensives. Based on the above effects of the combined use, the compositions described herein can inhibit the increase in heart rate.

The compositions useful for treating cardiovascular diseases of the present invention can be administered as an alternative to subjects who have been undergoing an antihypertensive therapy with calcium channel blocker(s) other than cilnidipine and an angiotensin II receptor blocker(s). In particular, the compositions can decrease blood pressure better than when using the antihypertensive therapy.

Specifically, examples of the calcium channel blockers preferably include dihydropyridine calcium channel blockers other than cilnidipine. More specifically, examples include amlodipine, nifedipine, benidipine, nilvadipine, manidipine, azelnidipine, felodipine, nisoldipine, nitrendipine, barnidipine, nicardipine and efonidipine. Amlodipine and nifedipine are preferable, and nifedipine is particularly preferable.

When the calcium channel blocker is nifedipine, the compositions of the present invention can not only decrease blood pressure better than the antihypertensive therapy, but also inhibit the increase in heart rate that occurs with the antihypertensive therapy.

When the compositions of the present invention are administered to a human being whose heart rate is 75 beats/min. or higher, and preferably 85 beats/min. or higher, the increase in heart rate typically caused by a decrease in blood pressure as a result of the administration is more effectively inhibited. Therefore, the heart rate is not increased, and is effectively stabilized.

When the subject is a human being, males are preferable. This is because there is a lower rate of the occurance of side-effects.

The cilnidipine and at least one angiotensin II receptor blocker may be formulated in advance together, or they may be separately formulated and then mixed when needed for administration, or they may be separately formulated and administered separately.

The compositions of the present invention may be in the form of a kit which contains a composition of cilnidipine and a composition of at least one angiotensin II receptor blocker.

The cilnidipine and at least one angiotensin II receptor blocker may be administered simultaneously, or one may be administered first, followed by the other if the effects of the combined use thereof, particularly synergetic effects can be obtained. When administered separately, the dosing interval between administration of each agent is not particularly limited.

In the present invention, cilnidipine and an angiotensin II receptor blocker(s) can be orally or parenterally administered, separately or simultaneously, and directly or by being mixed with pharmaceutically acceptable carriers, such as solid preparations, e.g. dispersants, granules, tablets and capsules; solutions, e.g. syrups, emulsions, injectable solutions (including those for subcutaneous, intravenous or intramuscular injection, and intravenous fluids); sublingual formulations; buccals; trochisci; microcapsules; sustained-release coated preparations; or suppositories. It is preferable to be orally administered in tablet form.

As for the pharmaceutically acceptable carriers, commonly used various organic or inorganic carriers can be used. In case of the solid preparations, excipients, lubricants, binders, disintegrating agents or the like are typically used, and in case of liquid preparations, solvents, solubilizing agents, suspending agents, tonicity agents, buffering agents, soothing agents or the like are typically used. If necessary, the additives such as antiseptic agents, antioxidant agents, coloring agents, sweetening agents, flavoring agents, or the like may be added.

Particularly, when simultaneously administering cilnidipine and at least one angiotensin II receptor blocker, it is preferable to be administered as a pharmaceutical composition containing cilnidipine and at least one angiotensin II receptor blocker(s). The dosage forms of the pharmaceutical composition can be those mentioned above.

The preparations of the above dosage forms can be produced in accordance with known preparation methods in the field.

The ratio of cilnidipine and at least one angiotensin II receptor blocker is within a range that results in the best effects of the combined use. For example, the at least one angiotensin II receptor blocker is usually 0.01 to 100, preferably 0.1 to 100, and more preferably 0.1 to 10 relative to 1 of cilnidipine, by weight. Especially, when using candesartan, candesartan is usually 0.01 to 100, preferably 0.05 to 50, and more preferably 0.05 to 5 relative to 1 of cilnidipine by weight ratio.

The subjects to which the compositions of the present invention are administered include mammals such as mice, rats, hamsters, rabbits, felines, canines, bovines, sheep, apes and human beings. Human beings are particularly preferable.

The administered dose per day of the compositions of the present invention differs depending on presented symptoms, age, sex, body weight, sensitivity to drugs, as well as the timing, interval, and route of administration. In general, the combined amount per a day by the oral administration is about 0.1 to 100 mg of cilnidipine and about 0.01 to 500 mg of an angiotensin II receptor blocker per 1 kg by body weight of a mammal. It is preferable that the combined amount is about 0.5 to 50 mg of cilnidipine and about 0.03 to 300 mg of an angiotensin II receptor blocker, and more preferably about 1 to 20 mg of cilnidipine and about 0.2 to 200 mg of an angiotensin II receptor blocker. If necessary, these amounts are administered in 1 to 3 divided doses.

The following non-limiting Examples further illustrate the present invention.

### EXAMPLES

A special investigation was conducted in various medical centers, compiled, and analyzed to evaluate the safety and availability of the combined use of cilnidipine and at least one angiotensin II receptor blocker.

Investigation Method

The investigation was conducted mainly in the departments of internal medicine/cardiovascular diseases of various medical institutions. The target of the estimated collected cases was about 3000 cases.

The subjects of the investigation were hypertensive patients which were adapted to cilnidipine. Combined administration of cilnidipine and ARB was started for the treatment of hypertension in these patients.

A prospective continuous investigation method was conducted. Such a method is continuously conducted with the registered cases with the combined administration of cilnidipine (Atelec®) and ARB, until the number of cases reaches the contracted cases for each center.

Regarding the administration method of cilnidipine, 5 to 10 mg of cilnidipine was orally administered after breakfast once a day. Meanwhile, the amount may increase or decrease depending on patient's age, symptoms, or the like. When the effect is insufficient, the amount can be increased to 20 mg once a day. The dosing period of cilnidipine was set to 12 weeks in principle.

The following items were tracked during the investigation: patient's background (sex), existence or nonexistence of previous therapeutic agents and the content thereof, targeted blood pressure (systolic BP/diastolic BP), the conditions of administration of cilnidipine and ARB, antihypertensives used in combination (those other than cilnidipine or ARB), clinical course (systolic BP/diastolic BP/heart rate), and occurrence of side-effects. As for the availability of the combined administration, in the 12th week after starting the combined administration, the doctor in attendance judged the degree of the blood pressure controls on a three-point scale of “well-controlled”, “nearly well-controlled” and “poorly-controlled”, referring to the changes in the systolic and diastolic BP. As for the safety and availability analysis for each patient background, Fisher's exact probability test or the X² test was used. As for the blood pressure and heart rate, the average value and the standard deviation were calculated for the subjects before the combined administration (within 4 weeks before starting the combined administration), and data after starting the combined administration were determined on each observation day, and analysis was conducted by using a paired t-test. The test was conducted as the two-sided test and less than 5% of the risk rate was regarded as significant. Furthermore, side-effects were compiled based on the naming of preferred terms (PT) of MedDRA/J ver 8.0.

Results

1. Compilation of Cases from Medical Centers

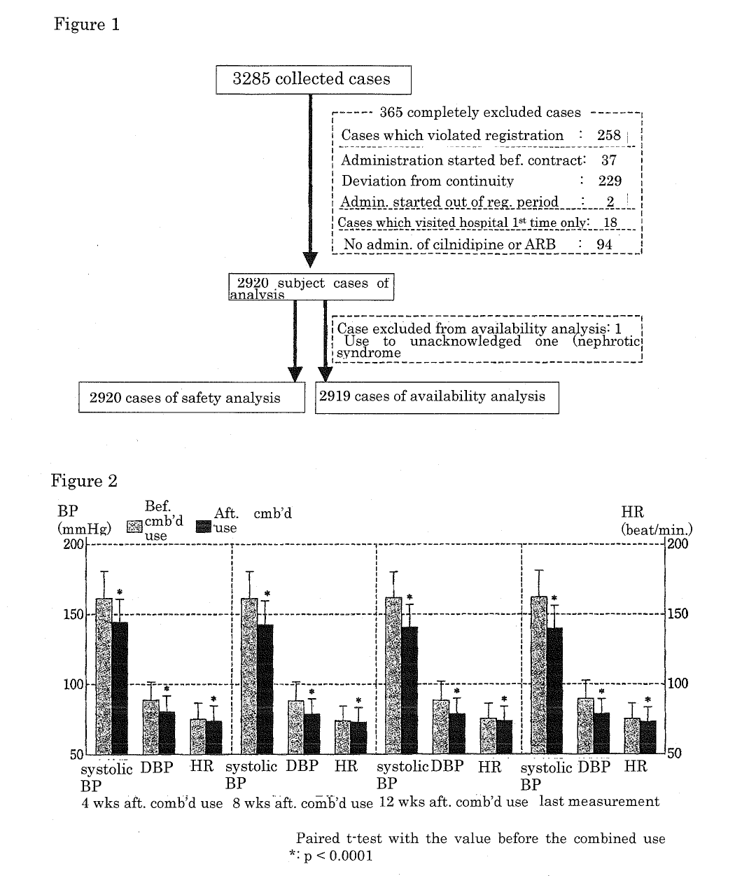

3322 cases in 471 centers throughout the country of Japan were randomly registered in accordance with the continuous investigation method, and the survey sheets of 3285 cases were collected (37 cases could not be collected). FIG. 1 shows the 3285 collected cases. Among these collected cases, a total of 365 cases were excluded for the following reasons (2 or more reasons apply to 11 cases): 258 cases violated registration, 18 cases could not be determined since the subject did not visit the hospital at all after staring the combined therapy, and 94 cases did not receive cilnidipine or ARB. Thus, 2920 cases were assigned to the safety analysis. Furthermore, one case consisted of a patient with nephrotic syndrome, which was not acknowledged, and so was excluded from the 2920 cases. Therefore, 2919 cases were assigned to the availability analysis.

2. Background of Patients

1) Table 1 shows the patients' background for the 2920 cases used in the safety analysis.

TABLE 1
Patients' background for cases in the safety analysis:
Attributes of cases:

| Items | | Case No. | ratio (%) |
|---|---|---|---|
| Sex | Male | 1,416 | 48.5 |
| | Female | 1,481 | 50.7 |
| | Unknown | 23 | 0.8 |
| Antihypertensives | None | 422 | 14.5 |
| before starting the | Taking | 2,483 | 85.0 |
| combined | Unknown | 15 | 0.5 |
| administration | | | |
| Antihypertensives used in | None | 2,185 | 74.8 |
| combination other than | Taking | 735 | 25.2 |
| cilnidipine or ARB | | | |
| Max. administered | less than 10 mg | 389 | 13.3 |
| dose per a day of | 10 mg | 2,175 | 74.5 |
| cilnidipine | over 10 mg | 345 | 11.8 |
| | Unknown | 11 | 0.4 |
| Total | | 2,920 | |

There were 1416 males (48.5%) and 1481 females (50.7%).

The cases undergoing hypertension treatment with antihypertensives prior to the start of this investigation were 2483 (85.0%). These hypertension treatments included ARB, calcium channel blockers, or the like. Among these, the cases which consisted of sole administration of ARB were 1179 (47.5%); the cases which consisted of ARB and other antihypertensives administered in combination were 944 (38.0%). Among these, the cases which consisted of ARB and calcium channel blockers administered in combination were 684 (23.4%), and the cases which consisted of ARB administered before administration of cilnidipine were 2123. After starting this investigation, all cases were administered ARB and cilnidipine in combination. The administration methods of cilnidipine were (1) administration of cilnidipine after administration of ARB alone; (2) treatment with calcium channel blockers and ARB, and then switching to cilnidipine and ARB; (3) administration of ARB and cilnidipine simultaneously, and the like.

The antihypertensives other than cilnidipine or ARB were administered in combination at the rate of 25.2%.

As for the maximum administered dose per a day of cilnidipine, less than 10 mg were 3 89 cases (13.3%), 10 mg were 2175 cases (74.5%) and over 10 mg were 345 cases (11.8%). 87.8% of the cases were within the range of usual dosage and administration.

In the safety analysis, the case that was excluded from the availability analysis due to the patient's background had the following attributes:

sex: female

antihypertensives before starting the combined use: losartan

antihypertensive used in combination other than cilnidipine or ARB: none

maximum administered dose per day of cilnidipine: 10 mg

ARB used in the combined use: losartan,

and cilnidipine was additionally administered to this case after the administration of ARB alone.

2) Table 2 shows the kind of ARB used in combination with cilnidipine and the average administered dose thereof per day for the 2919 cases in the availability analysis. As for the maximum administered dose of candesartan per day when starting the combined administration, 281 cases (27.7%) were less than 8 mg, 693 cases (68.3%) were 8 mg and 37 cases (3.6%) were over 8 mg.

TABLE 2
(The kind of ARB and the average administered dose
per day in the cases in the availability analysis)

administered dose (mg)*

| | start admin. | last meas. |
|---|---|---|
| candesartan | 7.06 ± 2.49 | 7.41 ± 2.51 |
| | n = 1015 | n = 1006 |
| valsartan | 79.67 ± 26.48 | 82.27 ± 29.14 |
| | n = 1101 | n = 1098 |
| losartan | 49.36 ± 19.40 | 51.59 ± 20.22 |
| | n = 548 | n = 523 |
| telmisartan | 37.39 ± 11.41 | 39.01 ± 12.53 |
| | n = 244 | n = 271 |
| olmesartan | 18.18 ± 4.05 | 17.78 ± 4.28 |
| | n = 11 | n = 18 |

*average ± standard deviation

3) Table 3 shows the kind of ARB when starting the combined administration and the average administered dose thereof for 1178 cases in the availability analysis, which were undergoing hypertension treatment with administration of ARB alone, and the cases to which cilnidipine was singularly and additionally administered, and antihypertensives other than ARB or cilnidipine were not administered during the treatment period.

TABLE 3
(The kind of ARB and the average administered dose per day
in the cases which were administered ARB alone, and then
cilnidipine was singularly and additionally administered)

administered dose (mg)*

| | start admin. | last meas. |
|---|---|---|
| candesartan | 7.09 ± 2.91 | 7.46 ± 2.90 |
| | n = 422 | n = 420 |
| valsartan | 77.57 ± 20.83 | 80.64 ± 24.18 |
| | n = 430 | n = 427 |
| losartan | 48.47 ± 16.44 | 49.58 ± 17.16 |
| | n = 223 | n = 215 |
| telmisartan | 36.63 ± 7.52 | 38.23 ± 9.84 |
| | n = 101 | n = 113 |
| olmesartan | 20.00 ± 0.00 | 20.00 ± 0.00 |
| | n = 2 | n = 18 |

*average ± standard deviation

4) Table 4 shows the kind of ARB used in the combined administration with cilnidipine and the average administered dose thereof in the 684 cases in the availability analysis, which were undergoing hypertension treatment with the combined administration of ARB and a calcium channel blocker other than cilnidipine, and the cases wherein the calcium channel blocker was switched to cilnidipine. Meanwhile, examples of the calcium channel blockers other than cilnidipine which were used before switching are the pharmaceutical compositions which are acknowledged as antihypertensives, such as amlodipine, nifedipine, benidipine, nilvadipine, manidipine, azelnidipine, felodipine, nisoldipine, nitrendipine, barnidipine, nicardipine and efonidipine. The administration method thereof is the acknowledged dosage and administration.

TABLE 4
(The kind of ARB and the average administered dose per day in the
cases wherein administration of ARB + a calcium channel blocker
other than cilnidipine was switched to that of ARB + cilnidipine)

administered dose (mg)*

| | start admin. | last meas. |
|---|---|---|
| candesartan | 7.30 ± 2.05 | 7.55 ± 2.13 |
| | n = 208 | n = 206 |
| valsartan | 81.84 ± 31.91 | 84.52 ± 34.52 |
| | n = 278 | n = 285 |
| losartan | 49.05 ± 20.31 | 53.23 ± 21.64 |
| | n = 142 | n = 127 |
| telmisartan | 39.44 ± 16.98 | 40.18 ± 16.09 |
| | n = 55 | n = 58 |
| olmesartan | 20.00 ± 0.00 | 20.00 ± 0.00 |
| | n = 1 | n = 1 |

*average ± standard deviation

3. Safety

The ratio of the cases wherein side-effects occurred was low and any specific tendency was not seen. However, the ratios of the cases wherein side-effects occurred were significantly different between the sexes as mentioned below.

1) Conditions of the Occurrence of Side-Effects by Sex

The ratios of the cases wherein side-effects occurred were compared and examined between the sexes in the 2920 cases of the safety analysis (Table 5). As a result, the ratios of the cases wherein side-effects occurred were significantly different between the sexes. The ratio thereof in a female group was 3.2% and that in a male group was 1.8%. Thus, the ratio of the cases wherein side-effects occurred was significantly lower in the male group. The side-effects observed were mainly subjective symptoms such as headache, palpitation, and free-floating dizziness. Meanwhile, the ratio of the cases wherein the side-effects of these subjective symptoms occurred was low, and it is not a serious side-effect caused by the combined administration.

TABLE 5

| | | side-effect | | | |
|---|---|---|---|---|---|
| | number | number of | number of | occurence | test |
| | of cases | examples | cases | rate (%) | result |
| male | 1,416 | 26 | 32 | 1.84 | p = 0.0240 |
| female | 1,481 | 47 | 56 | 3.17 | |
| unknown | 23 | 0 | 0 | 0 | |

4. Availability

1) Degree of the Blood Pressure Controls

As the index of availability, in the 12th week after starting the combined administration, the doctor in attendance judged the degree of the blood pressure controls on a three-point scale of “well-controlled”, “nearly well-controlled” and “poorly-controlled”.

(1) Among the 2919 cases in the availability analysis, 42 cases in which the degree of the blood pressure controls was not described, and 168 cases which could not be judged, were excluded. In the other 2709 cases, the ratio of number of cases judged as “nearly well-controlled” or higher was calculated as the efficacy ratio. Accordingly, the efficacy ratio of the subject cases in the availability analysis was 84.8% (2298 cases/2709 cases).

(2) Furthermore, in order to remove the effect of the combined use of cilnidipine and a antihypertensive other than ARB, and to clarify the effect of the combined use of cilnidipine and ARB, in the cases of the availability analysis, the efficacy ratio of the cases undergoing hypertension treatment with administration of ARB alone, and the cases wherein cilnidipine was additionally administered (antihypertensives are ARB and cilnidipine only), the ratio was 86.4% (1018 cases/i 178 cases). As for the side-effects, the ratio of the cases wherein side-effects occurred was low and any specific tendency was not seen in the cases which were undergoing treatment with the administration of ARB alone, and said cases in which cilnidipine was additionally administered.)

(3) Furthermore, among the cases which were undergoing hypertension treatment with the administration of ARB alone, and the cases in which cilnidipine was singularly and additionally administered, Table 6 shows the average administered dose per day for each kind of ARB and the efficacy ratio (based on the degree of the blood pressure controls in the 12th week as mentioned above) of the cases in which the same ARB was continuously administered without changing the kind of the drug from the start of the combined administration to the 12th week and beyond, until the last measurement.

TABLE 6
(Efficacy ratio in the cases to which only ARB + cilnidipine was additionally
administered (the kind of ARB agent unchanged)

| | | | | cilnidipine ave. | |
|---|---|---|---|---|---|
| | no. of | ARB ave. admin'd dose (mg)* | | admin'd dose (mg)* | efficacy |
| | cases | admin. start | 12^{th} wk to last meas. | 12^{th} wk to last meas. | ratio (%) |
| candesartan | 379 | 7.15 ± 3.00 | 7.48 ± 2.97 | 9.66 ± 2.96 | 85.5 |
| valsartan | 393 | 77.28 ± 20.56 | 80.25 ± 23.34 | 9.48 ± 2.72 | 86.0 |
| losartan | 196 | 48.12 ± 15.72 | 49.53 ± 16.72 | 9.05 ± 3.17 | 89.8 |
| telmisartan | 92 | 36.52 ± 7.62 | 37.61 ± 8.30 | 10.27 ± 3.28 | 88.0 |
| olmesartan | 2 | 20.00 | 20.00 | 7.50 ± 3.54 | 100.0 |

*average ± standard deviation

2) Changes in Blood Pressure and Heart Rate

(1) All cases in the availability analysis

Table 7 and FIG. 2 show the number of cases for which the blood pressure was measured, and the changes in the measured values of systolic BP, diastolic BP, and heart rate were measured at the following time points: before starting the combined administration, 4 weeks, 8 weeks, and 12 weeks after starting the combined administration, and the last measurement, which was 222.43 days in average from the start of the combined administration, n=2919). The blood pressure after starting the combined administration significantly decreased in both systolic and diastolic BP at 4 weeks. Then, it continued to slowly decrease, and the a stable change was seen up to 12 weeks after the start. The heart rate after starting the combined administration significantly decreased at 4 weeks, and a continued decrease was seen up to 12 weeks after the start.

TABLE 7
Change in BP and HR in the cases in
the availability analysis

| | before combined use | | 4 wks aft. use | 8 wks aft. use | 12 wks aft. use | last meas. |
|---|---|---|---|---|---|---|
| systolic | bef. | 162.21 ± 19.21 | 161.96 ± 19.46 | 161.13 ± 18.47 | 161.63 ± 18.58 | 162.19 ± 19.11 |
| BP | aft. | — | 144.31 ± 16.64* | 142.28 ± 17.41* | 140.57 ± 16.45* | 139.67 ± 16.47* |
| (mmHg) | cases | 2,586 | 1,131 | 840 | 950 | 2,565 |
| diastolic | bef. | 89.49 ± 13.09 | 88.80 ± 12.94 | 88.22 ± 13.53 | 88.56 ± 13.48 | 89.51 ± 13.09 |
| BP | aft. | — | 80.44 ± 11.30* | 78.69 ± 10.99* | 78.64 ± 11.39* | 78.78 ± 10.68* |
| (mmHg) | cases | 2,586 | 1,131 | 840 | 950 | 2,565 |
| heart | bef. | 75.40 ± 11.47 | 75.06 ± 11.46 | 73.83 ± 10.49 | 75.33 ± 10.98 | 75.18 ± 11.30 |
| rate | aft. | — | 73.80 ± 10.80* | 72.87 ± 10.53* | 73.61 ± 10.57* | 73.18 ± 10.37* |
| (beat/ | cases | 1,815 | 765 | 557 | 664 | 1,615 |
| min.) | | | | | | |

Paired t-test with the value before the combined use
*p < 0.0001
Cases indicate the number of examples upon each measurement and before the combined use.

(2) Cases in which only ARB was administered and then cilnidipine was singularly and additionally administered

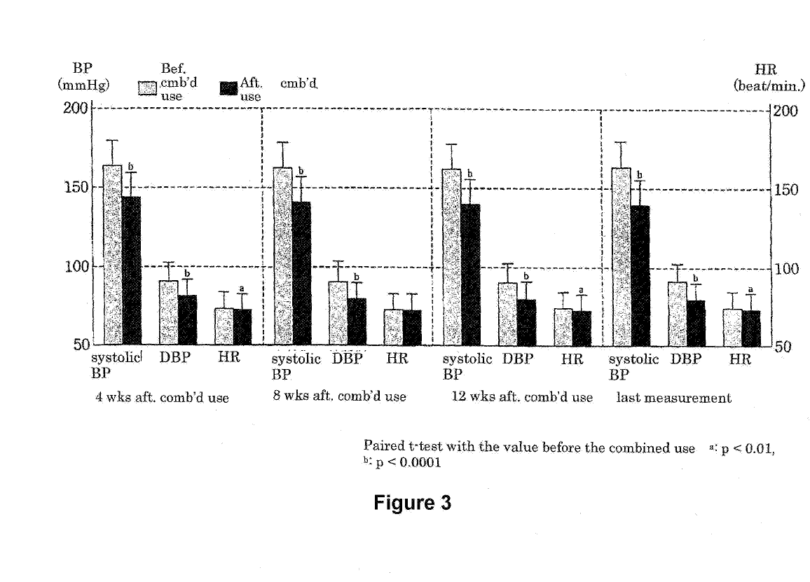

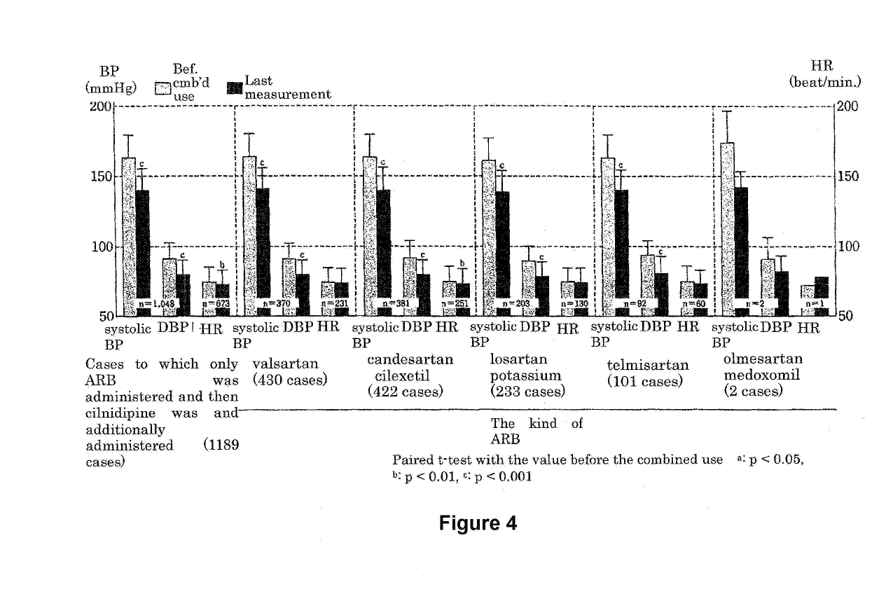

The changes in blood pressure and heart rate were examined for the cases which were undergoing hypertension treatment with administration of ARB alone, and the cases in which cilnidipine was singularly and additionally administered (Antihypertensives are ARB and cilnidipine only. The 12th week and up to the last measurement is 222.03±143.88 days after starting the combined administration (average±standard deviation), n=1178). As shown in Table 8 and FIG. 3, a significant decrease was seen at 4 weeks in systolic BP, diastolic BP, and heart rate. The same stable hypotensive effect was seen 12 weeks and later, up to the last measurement in all cases.

TABLE 8
Transition of BP and HR of the cases to which only ARB was administered and
then cilnidipine was singularly and additionally administered

| | | | | 8 wks after | | |
|---|---|---|---|---|---|---|
| | before combined use | | 4 wks after the use | the use | 12 wks aft the use | last measurement |
| systolic | bef. use | 163.09 ± 16.36 | 163.61 ± 15.91 | 162.45 ± 16.04 | 161.92 ± 15.66 | 163.11 ± 16.40 |
| BP | aft. use | — | 143.91 ± 15.39^{b} | 140.97 ± 15.94^{b} | 140.09 ± 15.37^{b} | 139.73 ± 15.77^{b} |
| (mmHg) | cases | 1,055 | 453 | 338 | 360 | 1,047 |
| diastolic | bef. use | 91.26 ± 11.48 | 90.72 ± 12.00 | 90.63 ± 13.13 | 90.26 ± 12.40 | 91.31 ± 11.47 |
| BP | aft. use | — | 81.47 ± 10.51^{b} | 79.75 ± 10.37^{b} | 79.71 ± 11.23^{b} | 79.68 ± 10.68^{b} |
| (mmHg) | cases | 1,055 | 453 | 338 | 360 | 1,047 |
| heart | bef. use | 74.46 ± 10.76 | 73.40 ± 10.54 | 72.97 ± 10.16 | 73.98 ± 10.44 | 74.26 ± 10.58 |
| rate | aft. use | — | 72.49 ± 10.25^{a} | 72.62 ± 10.60 | 72.54 ± 10.28^{a} | 73.24 ± 10.51^{a} |
| (beat/ | cases | 738 | 308 | 229 | 249 | 648 |
| min.) | | | | | | |

Paired t-test with the value before the combined use
^{a}p < 0.01,
^{b}p < 0.0001
Cases indicate the number of examples upon each measurement and before the combined use.

(3) Cases in which only an ARB was administered and then cilnidipine was singularly and additionally administered (per ARB agents)

i) FIG. 4 shows the changes in blood pressure and heart rate before starting the combined administration, and 12 weeks after the start, and up to the last measurement, per each ARB agent, for the patients which were undergoing hypertension treatment with administration of ARB alone, before starting the administration of cilnidipine and said patients to which cilnidipine was singularly and additionally administered (antihypertensives are ARB and cilnidipine only). A significant decrease was seen in systolic BP and diastolic BP 12 weeks and later up to the last measurement as compared with those before starting the combined use with cilnidipine. The heart rate significantly decreased when using candesartan (p<0.01).

ii) Furthermore, analysis was conducted only in the cases wherein the kind and administered dose of ARB and the administered dose of cilnidipine were unchanged from the start to the end of the test. Table 9 shows the administered dose and the results thereof. According to the paired t-test with the value before the combined use, a significant decrease was seen (p <0.001) notwithstanding the kind of ARB, in systolic BP and diastolic BP 12 weeks or later to the last measurement as compared with those before starting the combined use with cilnidipine. The heart rate significantly decreased when using candesartan (p<0.01).

TABLE 9
Change in BP and HR for each ARB agent of the cases to which only ARB was administered and
then clinidipine was singularly and additionally administered (not including other
antihypertensives, administered dose unchanged)

| | admin'd dose | | | | | | | |
|---|---|---|---|---|---|---|---|---|
| | (mg) | | diastolic BP (mmHg) | | systolic BP (mmHg) | | heart rate (beat/min.) | |
| | ARB | cilnidipine | start admin. | last meas.* | start admin. | last meas.* | start admin. | last meas. |
| candesartan | 8 | 10 | 164.98 ± 16.35 | 141.02 ± 17.36 | 89.59 ± 14.80 | 79.81 ± 12.72 | 74.19 ± 10.96 | 72.47 ± 11.14 |
| | | | n = 193 | | | | n = 129 | |
| valsartan | 80 | | 160.65 ± 16.04 | 140.75 ± 15.61 | 91.43 ± 11.20 | 80.62 ± 10.40 | 74.08 ± 10.41 | 72.89 ± 10.20 |
| | | | n = 245 | | | | n = 146 | |
| losartan | 50 | | 161.84 ± 16.13 | 137.74 ± 15.19 | 90.40 ± 10.35 | 79.32 ± 9.82 | 74.13 ± 8.98 | 73.66 ± 9.49 |
| | | | n = 99 | | | | n = 62 | |
| telmisartan | 40 | | 163.00 ± 17.32 | 138.89 ± 15.89 | 93.52 ± 10.84 | 80.49 ± 12.78 | 74.24 ± 11.55 | 72.12 ± 9.42 |

| | |
|---|---|
| n = 61 | n = 41 |

*211.43 ± 133.43 day after starting the combined use (average ± standard deviation), n = 598

iii) The results 12 weeks after starting the combined administration are shown herewith. Table 10 shows the changes in blood pressure and heart rate before starting the combined administration and 12 weeks after the start, for each ARB agent, for the patients undergoing hypertension treatment with administration of ARB alone, before starting the administration of cilnidipine, and patients to which cilnidipine was additionally administered (antihypertensives are ARB and cilnidipine only). A significant decrease was seen (p<0.0001) notwithstanding the kind of ARB, in systolic BP and diastolic BP measured in the 12th week as compared with those before starting the combined use of cilnidipine. The heart rate significantly decreased when using candesartan (p<0.01).

TABLE 10

| | diastolic BP (mmHg) | | systolic BP (mmHg) | | heart rate (beat/min.) | |
|---|---|---|---|---|---|---|
| | upon start. admin. | 12^{th} wk aft admin. | upon start. admin. | 12^{th} wk aft admin. | upon start. admin. | 12^{th} wk aft admin. |
| candesartan | 163.78 ± 17.52 | 141.29 ± 17.02 | 89.59 ± 14.80 | 79.81 ± 12.72 | 75.14 ± 11.48 | 72.66 ± 11.86 |
| | n = 133 | | | | n = 92 | |
| valsartan | 160.65 ± 15.20 | 138.32 ± 14.28 | 91.50 ± 10.58 | 79.31 ± 10.42 | 73.22 ± 9.51 | 71.65 ± 9.38 |
| | n = 124 | | | | n = 98 | |
| losartan | 160.78 ± 14.13 | 139.52 ± 14.43 | 88.67 ± 9.91 | 79.45 ± 9.20 | 73.81 ± 8.29 | 73.95 ± 7.68 |
| | n = 89 | | | | n = 62 | |
| telmisartan | 161.33 ± 12.62 | 143.64 ± 12.53 | 90.94 ± 12.27 | 80.70 ± 11.71 | 74.95 ± 12.70 | 73.09 ± 9.29 |

| | |
|---|---|
| n = 33 | n = 22 |

(4) Cases wherein ARB and another calcium channel blocker were administered, and then the calcium channel blocker was switched to cilnidipine, and combined administration with ARB was continued.

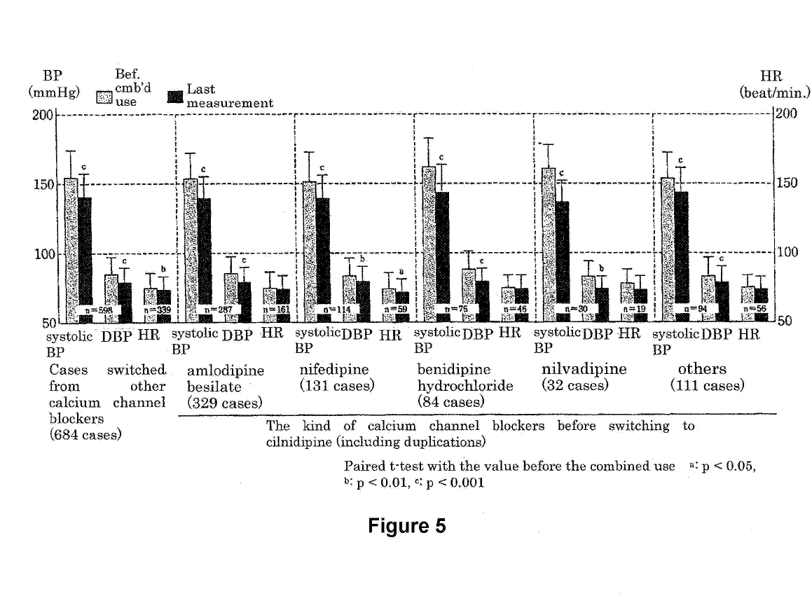

The cases which were undergoing hypertension treatment with the combined use of ARB and a calcium channel blocker other than cilnidipine before starting the investigation, and said cases wherein the calcium channel blocker was switched to cilnidipine, were 684 cases in the availability analysis. FIG. 5 shows the changes in blood pressure and heart rate for these cases.

The blood pressure 12 weeks and later, up to the last measurement in these switched cases significantly decreased as compared with that before switching. The systolic BP and diastolic BP per each calcium channel blockers before switching also significantly decreased notwithstanding the kind of calcium channel blockers. Furthermore, a significant decrease was seen in the heart rate of the whole cases wherein the other calcium channel blockers were switched and those wherein nifedipine was switched.

Table 11 shows the changes in blood pressure, heart rate, and the average administered dose per day for each ARB agent wherein the administration of 5 mg of amlodipine was switched to that of 10 mg of cilnidipine for the cases wherein the administered dose of each of ARB and cilnidipine was unchanged from the start their combined administration to 12 weeks or later to the last measurement.

TABLE 11

| | | | admin'd dose (mg/day) | | diastolic BP (mmHg) | | systolic BP (mmHg) | | heart rate (beat/min.) | |
|---|---|---|---|---|---|---|---|---|---|---|
| ARB | Cases | | start. admin. | last meas. | start. admin. | last meas. | start. admin. | last meas. | start. admin. | last meas. |
| valsartan | 62 | ave. | 76.23 | 76.23 | 158.15 | 140.67 | 89.36 | 80.74 | 77.18 | 75.86 |
| | | SD | 22.22 | 22.22 | 18.12 | 16.77 | 11.17 | 11.39 | 9.94 | 11.76 |
| | | | | | n = 61 | | | | n = 28 | |
| losartan | 23 | ave. | 52.17 | 52.17 | 150.35 | 138.22 | 82.00 | 79.78 | 72.00 | 72.44 |
| | | SD | 16.71 | 16.71 | 15.74 | 15.69 | 12.63 | 10.52 | 10.92 | 9.76 |
| | | | | | n = 23 | | | | n = 16 | |
| candesartan | 55 | ave. | 7.05 | 7.05 | 152.10 | 140.96 | 82.90 | 78.58 | 75.54 | 72.58 |
| | | SD | 1.99 | 1.99 | 17.95 | 16.72 | 13.12 | 13.36 | 12.51 | 6.63 |
| | | | | | n = 52 | | | | n = 24 | |
| telmisartan | 13 | ave. | 40.00 | 40.00 | 155.00 | 147.23 | 86.15 | 83.08 | 70.00 | 71.00 |
| | | SD | 14.14 | 14.14 | 22.46 | 17.02 | 15.63 | 10.58 | 8.19 | 5.79 |

| | |
|---|---|
| n = 13 | n = 10 |

The systolic BP and diastolic BP decreased notwithstanding the kind of ARB 12 weeks and later, up to the last measurement, and significant differences were seen in valsartan (P<0.0001), candesartan (P<0.01) and losartan (P<0.05).

3) Changes in Heart Rates as Compared to the Heart Rates Before Starting the Combined Administration

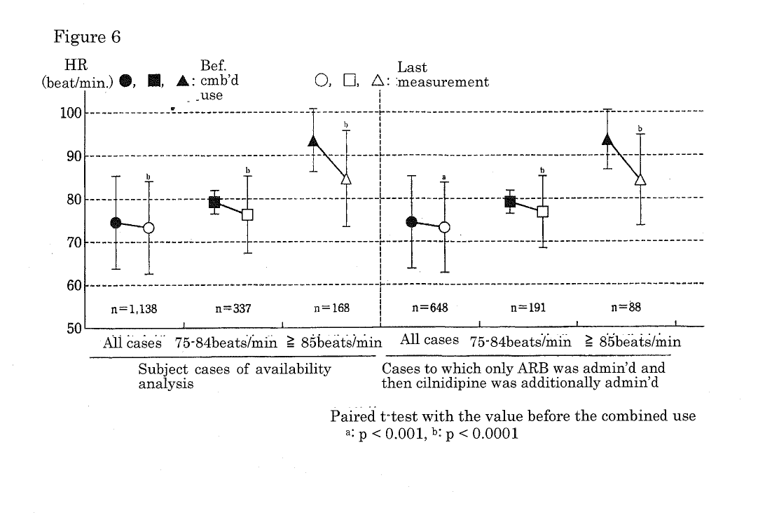

FIG. 6 shows the heart rates before starting the combined use and 12 weeks and later, up to the last measurement for all subject cases in the availability analysis and the cases to which only ARB was administered and then cilnidipine was additionally administered. Among all subject cases in the availability analysis, in the cases with heart rates of 75 beats/min. or higher and less than 85 beats/min. before starting the combined use, the heart rates after starting the combined administration significantly decreased from 79.19±2.73 beats/min. to 76.28±8.93 beats/min. In the cases with heart rates of 85 beats/min. or higher, the heart rates significantly decreased from 93.48±7.28 beats/min. to 84.57±11.17 beats/min. Furthermore, among the cases wherein only ARB was administered and then cilnidipine was additionally administered, in the cases with heart rates of 75 beats/min. or higher and less than 85 beats/min. before starting the combined use, the heart rates after starting the combined use significantly decreased from 79.24±2.71 beats/min. to 76.87±8.37 beats/min. In the cases with heart rates of 85 beats/min. or higher, the heart rates also significantly decreased from 93.64±6.90 beats/min. to 84.30±10.58 beats/min.

From above results, according to the present invention, the excellent hypotensive effect while at the same time inhibiting the increase in heart rate can be obtained by administering cilnidipine in combination with ARB. Therefore, the present invention is useful in preventing/treating cardiovascular diseases.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A composition useful for treatment or prevention of cardiovascular diseases comprising cilnidipine and at least one angiotensin II receptor blocker.

2. The composition according to claim 1, wherein said composition is formed by a process comprising separately formulating the cilnidipine and the at least one angiotensin II receptor blocker, and thereafter combining the cilnidipine and the at least one angiotensin II receptor blocker.

3. The composition according to claim 1, wherein the at least one angiotensin II receptor blocker is candesartan.

4. The composition according to claim 3, wherein the ratio of candesartan to cilnidipine is 0.05:1 to 5:1 by weight.

5. The composition of claim 1, wherein said composition has an antihypertensive effect when administered to a subject.

6. A method of treating or preventing cardiovascular diseases comprising administering an effective amount of the composition of claim 1 to a subject in need thereof.

7. The method of claim 6, wherein said subject has been undergoing an antihypertensive therapy with at least one calcium channel blocker other than cilnidipine, and at least one angiotensin II receptor blocker, and said administration of said composition is performed instead of administration of the at least one calcium channel blocker other than cilnidipine, and at least one angiotensin II receptor blocker.

8. The method according to claim 7, wherein the calcium channel blocker is nifedipine.

9. The method according to claim 6, wherein the subject is a human being with a heart rate of 75 beats/min. or higher.

10. The method according to claim 9, wherein the subject is male.

11. A method of treating or preventing a cardiovascular disease comprising administering an effective amount of a composition comprising cilnidipine and an effective amount of a composition comprising at least one angiotensin II receptor blocker to a subject in need thereof.

12. The method of claim 11, wherein said administration of a composition comprising cilnidipine and said administration of at least one angiotensin II receptor blocker is consecutive.

13. The method of claim 11, wherein said subject is a human having a heart rate of 75 beats/min. or higher.

14. The method of claim 13, wherein said subject is male.

15. The method of claim 11, wherein said at least one angiotensin II receptor blocker is candesartan.

16. The method according to claim 15, wherein the ratio of candesartan to cilnidipine is 0.05:1 to 5:1 by weight.

17. The method of claim 11, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

18. The method according to claim 17, wherein the calcium channel blocker is nifedipine.

19. A composition useful for treatment of hypertension comprising cilnidipine and at least one angiotensin II receptor blocker.

20. The composition according to claim 19, wherein said composition is formed by a process comprising separately formulating the cilnidipine and the at least one angiotensin II receptor blocker, and thereafter combining the cilnidipine and the angiotensin II receptor blocker.

21. The composition according to claim 19, wherein the at least one angiotensin II receptor blocker is candesartan.

22. The composition according to claim 21, wherein the ratio of candesartan to cilnidipine is 0.05:1 to 5:1 by weight.

23. The composition of claim 19, wherein said composition has an effect on cardiovascular diseases when administered to a subject.

24. A method of treating hypertension comprising administering an effective amount of the composition of claim 19 to a subject in need thereof.

25. The method of claim 24, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

26. The method according to claim 25, wherein the calcium channel blocker is nifedipine.

27. The method according to claim 24, wherein the subject is a human being with a heart rate of 75 beats/min. or higher.

28. The method according to claim 27, wherein the subject is male.

29. A method of treating hypertension comprising administering an effective amount of a composition comprising cilnidipine and an effective amount of a composition comprising at least one angiotensin II receptor blocker to a subject in need thereof.

30. The method of claim 29, wherein said administration of a composition comprising cilnidipine and said administration of at least one angiotensin II receptor blocker is consecutive.

31. The method of claim 29, wherein said subject is a human having a heart rate of 75 beats/min. or higher.

32. The method of claim 31, wherein said subject is male.

33. The method of claim 29, wherein said at least one angiotensin II receptor blocker is candesartan.

34. The method according to claim 33, wherein the ratio of candesartan to cilnidipine is 0.05:1 to 5:1 by weight.

35. The method of claim 29, wherein said subject has been undergoing an antihypertensive therapy with at least one calcium channel blocker other than cilnidipine, and at least one angiotensin II receptor blocker, and said administration of said composition is performed instead of administration of at least one calcium channel blocker other than cilnidipine, and at least one angiotensin II receptor blocker.

36. The method according to claim 35, wherein the calcium channel blocker is nifedipine.

37. A kit comprising a composition comprising cilnidipine and a composition comprising at least one angiotensin II receptor blocker.

38. The kit of claim 37, wherein said at least one angiotensin II receptor blocker is candesartan.

39. The kit of claim 38, wherein the ratio of candesartan to cilnidipine is 0.05:1 to 5:1 by weight.

40. A pharmaceutical composition comprising cilnidipine and candesartan as active ingredients.

41. The pharmaceutical composition according to claim 40, wherein the ratio of candesartan to cilnidipine is 0.05:1 to 5:1 by weight.
